(19) United States
(12) Patent Application Publication
(10) Pub. No.: US 2026/0248848 A1
(43) Pub. Date: Aug. 27, 2026

(54) EXTRACELLULAR VESICLES LOADED WITH POLYDOPAMINE NANOPARTICLES, AND PREPARATION METHOD

(71) Applicant: THE SECOND MEDICAL CENTER OF CHINESE PLA GENERAL HOSPITAL, Beijing (CN)

(72) Inventors: Feng CAO, Beijing (CN); Yang ZHANG, Beijing (CN); Zhongyang LIU, Beijing (CN)

(21) Appl. No.: 18/862,151

(22) PCT Filed: Sep. 7, 2023
(86) PCT No.: PCT/CN2023/117389

§ 371 Date: Oct. 31, 2024

(30) Foreign Application Priority Data
Sep. 7, 2022 (CN) 202211088658.3

(51) Int. Cl. A61K 35/15; A61K 9/50; A61K 31/785; C12N 5/00; C12N 5/0786

(52) U.S. Cl. CPC A61K 35/15; A61K 9/5068; A61K 9/5089; A61K 31/785; C12N 5/0018; C12N 5/0645; C12N 2501/2304; C12N 2501/231; C12N 2501/815

(57) ABSTRACT

The present disclosure discloses extracellular vesicles loaded with polydopamine nanoparticles, and a preparation method. A nano targeted treatment platform includes extracellular vesicles originating from M2 RAW264.7 macrophages and the polydopamine nanoparticles encapsulated within the extracellular vesicles as effective treatment ingredients. The nano targeted treatment platform of the present disclosure treats RAW264.7 macrophages with IL-4 and IL-10 cytokines to polarize them into M2 macrophages, and then co incubates polydopamine nanoparticles with the M2 macrophages to directly acquire extracellular vesicles loaded with the polydopamine nanoparticles using an extrusion method. The present disclosure may be applied in preparation of anti-inflammatory drugs or preparations for atherosclerosis. The present disclosure has a very objective application prospect in treatment of the atherosclerosis.

The present disclosure claims priority to Chinese Patent Application No. 2022110886583, filed on Sep. 7, 2022 and entitled “EXTRACELLULAR VESICLES LOADED WITH POLYDOPAMINE NANOPARTICLES AND PREPARATION METHOD” to the China National Intellectual Property Administration, the disclosure of which is herein incorporated by reference in its entirety.

### TECHNICAL FIELD

The present disclosure relates to a field of atherosclerosis treatment technologies and, in particular to, an extracellular vesicles-polydopamine nano targeted treatment platform, that is, extracellular vesicles loaded with polydopamine nanoparticles and a preparation method.

### BACKGROUND

The description of background of the present disclosure belongs to the related technology relevant to the present disclosure, which is merely used to illustrate and facilitate understanding of summary of the present disclosure, and should not be understood as applicants explicitly believing or inferring that the applicants believe that the present disclosure is the prior art on an application date of an initial application submission.

Atherosclerosis is a main cause of a cardiovascular disease, and the cardiovascular disease is one of main causes of global incidence rate and mortality. Inflammation is not only a critical indicator of the atherosclerosis, but also promotes progress of a whole disease. Therefore, anti-inflammation is considered as a promising strategy for treating the atherosclerosis.

However, anti-inflammatory drugs lack a specific targeted function towards inflammation sites, and their half-life is usually poor, which not only affects an actual treatment effect, but also produces many side effects and even leads to death. Moreover, nanomaterials with an anti-inflammatory effect are also limited in their application in an inflammation treatment due to its immunogenicity, toxicity, and biological distribution issues.

### SUMMARY

The present disclosure aims to provide extracellular vesicles loaded with polydopamine nanoparticles and a preparation method, which may make the size of nanovesicles controllable and a recovery rate high.

In order to solve the above problems in the prior art, the present disclosure provides the following technical solutions.

In a first aspect, the present disclosure provides a preparation method of extracellular vesicles loaded with polydopamine nanoparticles, including the following steps:
treating RAW264.7 macrophages with IL-4 and IL-10 cytokines to polarize them into M2 macrophages; and
after co incubating polydopamine nanoparticles with the M2macrophages, acquiring M2 macrophages-derived extracellular vesicles loaded with the polydopamine nanoparticles and with targeted inflammatory regions.

Further, the M2 macrophages-derived extracellular vesicles loaded with the polydopamine nanoparticles and with the targeted inflammatory regions are acquired by continuously extruding the M2 macrophages co incubated with the polydopamine nanoparticles using a size continuous extrusion method.

Further, the polydopamine nanoparticles are loaded onto the extracellular vesicles using the size continuous extrusion method.

Further, the M2 macrophages are acquired by treating the RAW264.7 macrophages with the IL-4 and IL-10 cytokines, and are co incubated with 100 ug/mL polydopamine nanoparticles in a cell culture incubator at a cell number of at least 1×10⁷ cells/ml.

Further, loading the polydopamine nanoparticles onto the extracellular vesicles specifically includes the following steps: after adding the polydopamine nanoparticles into an M2 macrophages culture system for co incubation, collecting cells using a cell scraper; using a micro extruder to sequentially extrude cell suspension through 1 μm, 400 nm, and 200 nm polycarbonate membranes; after continuously extruding five to six times, washing with PBS and centrifuging to remove unloaded polydopamine nanoparticles; and finally acquiring the extracellular vesicles loaded with the polydopamine nanoparticles.

In a second aspect, the present disclosure provides extracellular vesicles loaded with polydopamine nanoparticles, where the extracellular vesicles loaded with the polydopamine nanoparticles are prepared by the above preparation method.

Extracellular vesicles loaded with melatonin, where the extracellular vesicles loaded with the melatonin are prepared by the above preparation method.

Compared with the prior art, the present disclosure has the following advantages. Regarding to selection of nano raw materials for a method of the present invention, the M2 macrophages are selected as materials for a source of vesicles. The M2 macrophages themselves have an ability to target inflammatory environments and contain abundant anti-inflammatory factors, while the vesicles from which the M2 macrophages originate are not only rich in bioactive substances of parent cells, but also have an ability to actively target the inflammatory environments, as well as better biocompatibility and an immune evasion ability, enhancing a treatment effect.

In terms of the preparation method, the continuous extrusion method, as a physical method for acquiring the vesicles, is used to achieve the controllable size of the vesicles and a high acquisition rate, simplifying a process of acquiring the vesicles while reducing costs.

Functionally, nanovesicles can serve as carriers to deliver antioxidant polydopamine nanoparticles to inflammatory lesions, prolonging cycle time in vivo, meanwhile M2 macrophages-derived nanovesicles further have an anti-inflammatory effect. Combined with delivery of anti-inflammatory particles, a single delivery can achieve dual treatment effects.

The preparation method of the present disclosure is simple and effective, the M2 macrophages are easy to acquire, and the physical method of continuous extrusion is simple to operate, so that large-scale production may be achieved.

Further effects of the above non-conventional implementation manners will be explained combined with specific implementation manners in the following.

### BRIEF DESCRIPTION OF THE DRAWINGS

For clearer description of embodiments in the present disclosure or technical solutions in the prior art, drawings to be referred to for the description of the embodiments or the prior art are briefly introduced below. Apparently, the drawings in the description below merely illustrate some embodiments recorded in the present disclosure, and those skilled in the art may also derive other drawings according to these drawings without creative labors.

FIG. 1 shows a protein expression diagram of M2 macrophages acquired by co treating RAW 264.7 macrophages with IL-4 and IL-10 cytokines for 24 hours in extracellular vesicles loaded with polydopamine nanoparticles and a preparation method thereof of the present disclosure. That is, upregulation of protein marker Arg1 in the M2 macrophages and downregulation of protein marker iNOS in M1 macrophages observed through Western Blot experiments indicate successful acquisition of the M2 macrophages (Arg1: Arginase 1; iNOS: Inducible nitric oxide synthase; Actin: Actin; M0: Unsimulated activated macrophages; and M2: Alternative activated macrophages).

FIG. 2 shows a transmission electron microscopy diagram of passed polydopamine nanoparticles in extracellular vesicles loaded with polydopamine nanoparticles and a preparation method thereof of the present disclosure. Through a transmission electron microscopy, it is indicated that the polydopamine nanoparticles are spherical, with uniform size and distribution. This further indicates that the polydopamine nanoparticles are suitable for being loaded into extracellular vesicles that are also spherical in shape.

FIG. 3 shows extracellular vesicles loaded with polydopamine nanoparticles acquired by continuous size exclusion and continuous extrusion of a mixture of polydopamine nanoparticles co incubated with M2 macrophages in extracellular vesicles loaded with polydopamine nanoparticles and a preparation method thereof of the present disclosure. Through a transmission electron microscopy, it is indicated uniformity of the size of extracellular vesicles loaded with polydopamine nanoparticles. Through the transmission electron microscopy, it may be observed that a transparent bilayer lipid membrane structure is wrapped around the polydopamine nanoparticles, which are extracellular vesicles of a bilayer membrane structure, and inside vesicles are the loaded polydopamine nanoparticles.

FIG. 4 shows a confocal diagram of extracellular vesicles loaded with polydopamine nanoparticles exerting an antioxidant ability in extracellular vesicles loaded with polydopamine nanoparticles and a preparation method thereof of the present disclosure. Through cell experiments of intervention on macrophages, that is, foam cells, treated with H₂O₂ (1 mM) for 24 hours, it may be observed that the extracellular vesicles loaded with the polydopamine nanoparticles (PDA@M2 NVs) have excellent antioxidant properties. Through confocal microscopy observation of DCFH-DA, JC-1, and MitoSOx experiments, it may be observed that green signal values of DCFH-DA probes in PDA@M2 NVs treated cells decreased more significantly than polydopamine nanoparticles (PDA NPs) alone, indicating that reactive oxygen species content of the cells decreased after the intervention. Through JC-1 probe experiments, it may also be observed that PDA@M2 NVs treated red fluorescence signals are significantly higher than PDA NPs treated signal values, and at the same time, MitoSox red fluorescence signals are lower than PDA NPs in a group of PDA@M2 NVs, both of which may indicate that reactive oxygen species content of intracellular mitochondria is decreased more significantly after the intervention of PDA@M2 NVs. Therefore, it may be concluded that treatment of the extracellular vesicles loaded with the polydopamine nanoparticles can reduce reactive oxygen species of macrophages treated with H₂O₂ and exert an antioxidant function.

FIG. 5 is a fluorescence diagram of extracellular vesicles loaded with polydopamine nanoparticles actively targeting atherosclerotic plaque validation regions in extracellular vesicles loaded with polydopamine nanoparticles and a preparation method of the present disclosure. Through observation of fluorescence imaging of isolated blood vessels, it may be found that after tail vein injection of the extracellular vesicles loaded with the polydopamine nanoparticles, that is, PDA@M2 NVs, is completed, PDA@M2 NVs can be actively enriched into inflammatory regions of atherosclerotic plaques, that is, vessels are enriched with more intense fluorescent signals, with signal values that are significantly higher than enrichment of polydopamine nanoparticles alone on isolated atherosclerotic vessels, that is, an active targeted inflammation ability of PDA NPs (To ensure comparability of two groups, two groups of experiments use PDA NPs with DiD markers, so that signal values of the two groups are consistent, and at the same time, a dose of 20 mg/kg is used for tail vein injection for treatment and observation).

FIG. 6 shows a biological toxicity tissue slice diagram of extracellular vesicles loaded with polydopamine nanoparticles in extracellular vesicles loaded with polydopamine nanoparticles and a preparation method thereof of the present disclosure. Through HE staining, it may be observed that the extracellular vesicles loaded with the polydopamine nanoparticles have no significant toxic effect on internal organs of mice.

### DETAILED DESCRIPTION

Exemplary embodiments will be described in detail herein, with examples shown in drawings. When the following description refers to the drawings, same numbers in different drawings represent the same or similar elements unless otherwise indicated. Implementation manners described in the following exemplary embodiments do not represent all implementation manners consistent with the present disclosure. Rather, they are merely examples of apparatus and methods consistent with certain aspects of the present disclosure, as detailed in the appended claims.

Terms used in the present disclosure are merely for a purpose of describing specific embodiments and are not intended to limit the present disclosure. Singular forms of “one”, “the”, and “this” used in the present disclosure and the appended claims are also intended to include a majority form, unless context clearly indicates other meanings. It should also be understood that a term “and/or” used in the present disclosure refers to and includes any or all possible combinations of one or more associated listed items.

It should be understood that although terms first, second, third, and the like, may be used in the present disclosure to describe various information, the information should not be limited to these terms. These terms are merely used to distinguish information of the same type from each other. For example, without departing from a scope of the present disclosure, first information may also be referred to as second information, and similarly, the second information may also be referred to as the first information.

Applicants have found through extensive researches that extracellular vesicles are nano vesicles secreted by various types of cells, with excellent natural properties including outstanding biocompatibility, low cytotoxicity, immune inertness, specific targeting, and long-term circulation ability, making them effective drug delivery carriers. Exosomes derived from immune cells and mesenchymal stem cells have been successfully applied to encapsulate and deliver chemotherapeutic drugs, nucleic acid drugs, neurotransmitters and even nanoparticles for treating different diseases such as cancer and neurological disorders, but rarely for cardiovascular diseases. In addition, specificity accumulation of drug delivery systems based on extracellular vesicles is still limited, and even if they undergo targeted modifications and have targeted functions, some adverse reactions may still be caused due to different resistance such as formation of protein corona.

However, the extracellular vesicles, in addition to possessing their own unique properties, inherit specific content derived from parental cells (such as RNAs, DNAs, proteins, and small molecules), allowing them to graft natural biological functions of primitive cells and be directly used for treating diseases. M2 macrophages can secrete anti-inflammatory cytokines and still have an ability to target inflammatory regions after continuously extruding and encapsulating polydopamine nanoparticles with anti-inflammatory effects. A synergistic regulating effect of M2 macrophages-derived extracellular vesicles and an intracellular release of pre-loaded cargo dually promote M2 polarization of macrophages at lesions to regulate inflammation. A macrophage phenotype transition induced by M2 macrophages-derived extracellular vesicles has a good cell reprogramming ability and an innate biocompatibility, and by regulating alance between pro-inflammatory and anti-inflammatory macrophages, it may become a promising method for treating various inflammation related diseases. This work provides a strategy for designing modular extracellular vesicles that integrate advantages of naturally sourced extracellular vesicles and synthetic materials into various applications.

The applicants have found through extensive researches that inflammation plays an important role in occurrence and development of atherosclerotic plaques. In an early stage of atherosclerosis, causes of the atherosclerosis are endothelial damage, abnormal lipid metabolism, and hemodynamic damage. An atherosclerosis process is believed to be accompanied by inflammation changes of endothelial cells mediated by blood flow. When the endothelial cells are activated, they express inflammatory factors such as monocyte chemoattractant protein-1, intercellular adhesion molecule-1 (ICAM-1), vascular adhesion molecule-1 (VCAM-1), e-selectin, p-selectin, and the like, attract lymphocytes and monocytes that bind to endothelium and infiltrate artery walls, and the inflammation begins to occur. Many cells and cytokines are involved in this process, such as macrophages, lymphocytes (T and B cells), dendritic cells (DCs), endothelial cells (ECs), vascular smooth muscle cells (VSMCs), and tumor necrosis factor alpha (TNF-α). Therefore, in order to improve a treatment effect of anti-inflammatory polydopamine nanoparticles, enhance its targeting to atherosclerotic inflammatory lesion regions, improve its cycle time in vivo, and avoid being cleared by an immune system in vivo too quickly, use polarized M2 macrophages-derived extracellular vesicles to load polydopamine nanoparticles, inhibit the inflammatory factors, promote polarization of macrophages in lesion regions, and double play an anti-inflammatory effect, which may be crucial to improving the atherosclerosis.

The applicants have further found that extracellular vesicles (EVs) are natural microvesicles acquired from multiple types of cells, with excellent biocompatibility, low cytotoxicity, and immune inertness. Compared with common synthetic carriers, the extracellular vesicles are considered natural carriers for drugs and nanomaterials delivery applications due to their suitable nanoscale size and good biocompatibility, without causing adverse pro-inflammation and immune responses. In addition to their unique properties, EVs are further rich in specific components such as proteins, mRNAs, and miRNAs from mother cells. Therefore, they play a crucial role in various physiological and pathological processes to transplant natural biological functions of primitive cells, such as cell proliferation, differentiation, and viral transmission, which may be directly used to treat various diseases. In the present disclosure, the M2 macrophages are used as materials for a source of vesicles, that is, acquired extracellular vesicles can continue their natural chemotactic ability to target inflammatory regions, and inherit rich anti-inflammatory substances carried by M2 macrophages themselves, including various cytokines and nucleic acid substances. However, there are certain limitations in extraction, purification, and production efficiency of the extracellular vesicles at present. EVs of simulated nanovesicles (NVs) with similar size and composition to EVs are acquired by continuously extruding cells through a microfilter. The EVs acquired by extruding the cells can not only retain biological factors of stem cells themselves, but also greatly increase production of vesicles.

Based on the above findings, the applicants invented extracellular vesicles loaded with polydopamine nanoparticles for treating atherosclerosis and a preparation method thereof, which is specifically as follows.

A preparation method of extracellular vesicles loaded with polydopamine nanoparticles, including the following steps:
treating RAW264.7 macrophages with IL-4 and IL-10 cytokines to polarize them into M2 macrophages; and
after co incubating polydopamine nanoparticles with the M2 macrophages (adding 100 ug/ml polydopamine nanoparticles to an M2 macrophages culture system containing at least 1×10⁷ cells/ml and cultivating in a cell culture incubator for 12 hours), acquiring the M2 macrophages-derived extracellular vesicles loaded with the polydopamine nanoparticles and with the targeted inflammatory regions.

A preparation method of extracellular vesicles loaded with melatonin, including the following steps:
treating Raw264.7 macrophages with IL-4 and IL-10 cytokines to acquire M2 macrophages;
co-incubating polydopamine nanoparticles with the M2 macrophages;
acquiring extracellular vesicles loaded with polydopamine nanoparticles of controllable size and uniform size through size exclusion, that is, a continuous extrusion method, where nanoextracellular vesicles are derived from the M2 macrophages; and
after forming the extracellular vesicles loaded with polydopamine nanoparticles, removing free polydopamine nanoparticles by washing with PBS and centrifuging.

In some embodiments of the present disclosure, the extracellular vesicles of the controllable size are acquired by continuously extruding a mixture of polydopamine co incubated with polarized M2 macrophages using a size exclusion method.

In some embodiments, the preparation method of the extracellular vesicles loaded with the polydopamine nanoparticles includes the following steps:
(1) cultivating Raw264.7 macrophages;
(2) acquisition of M2 macrophages: co treating RAW264.7 macrophages with IL-4 (20 ng/ml) and IL-10 (10 ng/ml) cytokines for 24 hours;
(3) acquisition of nanovesicles loaded with polydopamine nanoparticles of M2 macrophages: after adding 100 ug/ml polydopamine nanoparticles to an M2 macrophages culture system containing at least 1×10⁷ cells/ml and cultivating in a cell culture incubator for 12 hours, aspirating supernatant and washing M2 macrophages three times with cold PBS to remove polydopamine nanoparticles that are not taken up by macrophages, collecting cells using a cell scraper; suspending the cells in PBS at a concentration of at least 1×10⁷ cells/ml; and using a small extruder (Avanti lipid extruder) to sequentially extrude cell suspension into 1 μm, 400 nm, and 200 nm polycarbonate membranes (Whatman Inc, USA) five to six times. Here, extruded vesicles are defined as nanovesicles (NVs), and the collected NVs are diluted in PBS and stored at −80° C.

M2 macrophages nanovesicles loaded with polydopamine nanoparticles, where extracellular vesicles loaded with the polydopamine nanoparticles are prepared by the above preparation method.

In acquisition steps of vesicles of the present disclosure, nanoparticles are directly cultured with cells, and suspension is subjected to continuous size exclusion, that is, mixed suspension of source cells and nanoparticles is continuously squeezed through different pores to directly acquire vesicles loaded with nanoparticles. This continuous extrusion process can be completed on a cell operating table, and an extrusion apparatus is lightweight and easy to hold. The amount of exosomes acquired may reach one microgram per microliter, up to 100 times the amount acquired by ultracentrifugation. At the same time, due to size exclusion, the size of acquired vesicles are mostly concentrated between 100 nm and 200 nm, achieving size controllability and having a certain spatial ability to load nanoparticles and maintain a long-term circulation ability in vivo. Moreover, a loading process is further simplified, with a one-step method to acquire the extracellular vesicles loaded with the polydopamine nanoparticles, without a need for a further ultrasound treatment for loading. In summary, technical problems to be solved by the present disclosure are to provide a large number of vesicles with the controllable size and an inflammation targeted function, which may be used for anti-inflammatory treatment of inflammatory regions of atherosclerotic plaques.

Combined with FIG. 1, through Western Blot experiments, it is observed protein expression of M2 macrophages acquired after treating RAW264.7 cells with IL-4 and IL-10 cytokines, that is, expression of M2 macrophages protein marker Arg1 is significantly increased, and expression of M1 macrophages protein marker iNOS is significantly decreased, indicating that M2 macrophages are successfully acquired.

Combined with FIG. 2, through a transmission electron microscopy, it is observed that polydopamine nanoparticles are spherical, with uniform size and distribution. This further indicates that the polydopamine nanoparticles are suitable for being loaded into extracellular vesicles that are also spherical in shape.

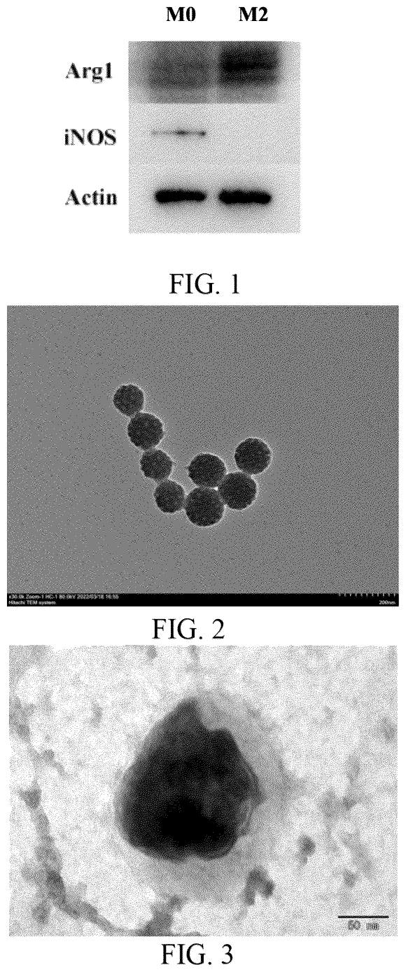

Combined with FIGS. 3, 100 ug/mL polydopamine nanoparticles are co incubated with M2 macrophages and subjected to continuous size exclusion and continuous extrusion, allowing the polydopamine nanoparticles to be directly loaded into M2 macrophages-derived extracellular vesicles. Due to gap extrusion of 1 μm, 400 nm, and 200 nm polycarbonate membranes, uniformity of their size is ensured, and a manufacturing process is simple. Through a transmission electron microscopy, it may be observed that a transparent bilayer lipid membrane structure is wrapped around the polydopamine nanoparticles, which are extracellular vesicles of a bilayer membrane structure, and inside vesicles are the loaded polydopamine nanoparticles.

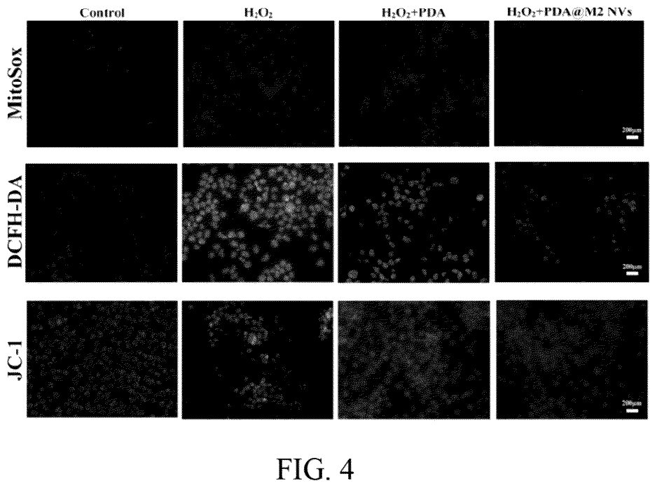

Combined with FIG. 4, it may be observed through cell experiments that extracellular vesicles loaded with polydopamine nanoparticles have excellent antioxidant properties. Through confocal microscopy observation of DCFH-DA, JC-1, and MitoSOx experiments, it may be clearly concluded that treatment of the extracellular vesicles loaded with the polydopamine nanoparticles can reduce reactive oxygen species of macrophages treated with H₂O₂ and exert an antioxidant function.

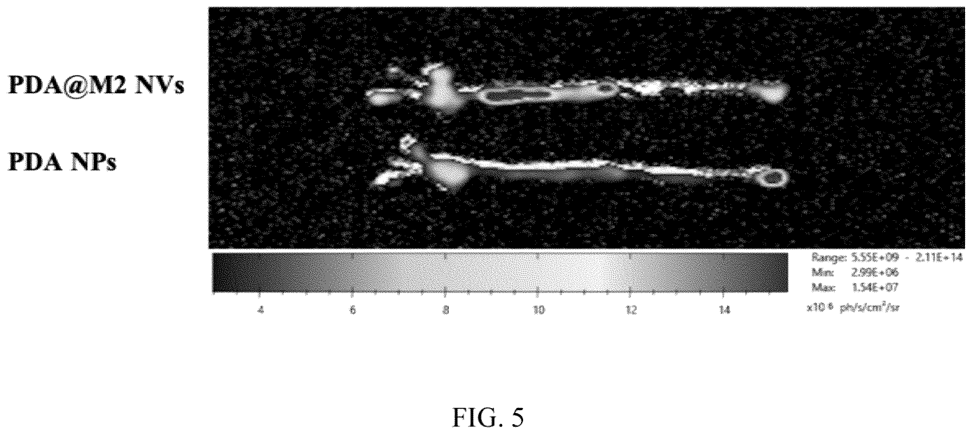

Combined with FIG. 5, through observation of fluorescence imaging of isolated blood vessels, after tail vein injection of extracellular vesicle loaded with polydopamine nanoparticles, that is, PDA@M2 NVs, is completed, PDA@M2 NVs can be actively enriched into inflammatory regions of atherosclerotic plaques, which is significantly superior to an active targeted inflammation ability of the polydopamine nanoparticles alone, that is, PDA NPs.

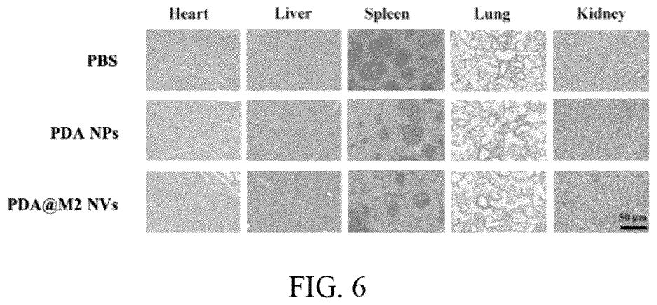

Combined with FIG. 6, after PDA@M2 NVs and PDA NPs are injected into mice via tail vein injection, a toxic effect on internal organs of the mice is observed, which may be observed by HE staining, and there is no obvious toxic effect.

The above are merely preferred specific implementation manners in the present disclosure, but a protection scope of the present disclosure is not limited thereto. Any variations or replacements that may be easily conceived of by those skilled that are familiar with the art within a technical scope disclosed in the present disclosure also fall within the protection scope of the present disclosure. Therefore, the protection scope of the present disclosure should be based on the protection scope of the claims.

The above are merely the embodiments of the present disclosure and are not intended to limit the present disclosure. For those skilled in the art, the present disclosure may have various modifications and changes. Any modifications, equivalent replacements, improvements, and the like made within spirits and principles of the present disclosure should fall within a scope of the claims of the present disclosure.

### INDUSTRIAL APPLICABILITY

The present disclosure provides extracellular vesicles loaded with polydopamine nanoparticles and a preparation method. Nanovesicles can serve as carriers to deliver antioxidant polydopamine nanoparticles to inflammatory lesions, prolonging cycle time in vivo, meanwhile M2 macrophages-derived nanovesicles further have an anti-inflammatory effect. Combined with delivery of anti-inflammatory particles, a dual therapeutic effect can be achieved with a single delivery. The preparation method of the present disclosure is simple and effective, the M2 macrophages are easy to acquire, and the physical method of continuous extrusion is simple to operate, so that large-scale production may be achieved. Formed products may be mass-produced and used in industry.

1: A preparation method of extracellular vesicles loaded with polydopamine nanoparticles, comprising the following steps:
treating RAW264.7 macrophages with IL-4 and IL-10 cytokines to polarize them into M2 macrophages; and
after co incubating polydopamine nanoparticles with the M2 macrophages, acquiring M2 macrophages-derived extracellular vesicles loaded with the polydopamine nanoparticles and with targeted inflammatory regions.

2: The preparation method of the extracellular vesicles loaded with the polydopamine nanoparticles according to claim 1, wherein the M2 macrophages-derived extracellular vesicles loaded with the polydopamine nanoparticles and with the targeted inflammatory regions are acquired by continuously extruding the M2 macrophages co incubated with the polydopamine nanoparticles using a size continuous extrusion method.

3: The preparation method of the extracellular vesicles loaded with the polydopamine nanoparticles according to claim 2, wherein the polydopamine nanoparticles are loaded onto the extracellular vesicles using the size continuous extrusion method.

4: The preparation method of the extracellular vesicles loaded with the polydopamine nanoparticles according to claim 1, wherein the M2 macrophages are acquired by treating the RAW264.7 macrophages with the IL-4 and IL-10 cytokines, and are co incubated with 100 ug/mL polydopamine nanoparticles in a cell culture incubator at a cell number of at least 1×10⁷ cells/ml.

5: The preparation method of the extracellular vesicles loaded with the polydopamine nanoparticles according to claim 1, wherein loading the polydopamine nanoparticles onto the extracellular vesicles specifically comprises the following steps: after adding the polydopamine nanoparticles into an M2 macrophages culture system for co incubation, collecting cells using a cell scraper; using a micro extruder to sequentially extrude cell suspension through 1 μm, 400 nm, and 200 nm polycarbonate membranes; after continuously extruding five to six times, washing with PBS and centrifuging to remove unloaded polydopamine nanoparticles; and finally acquiring the extracellular vesicles loaded with the polydopamine nanoparticles.

6: Extracellular vesicles loaded with polydopamine nanoparticles, wherein the extracellular vesicles loaded with the polydopamine nanoparticles are prepared by the preparation method according to claim 1.

7: Extracellular vesicles loaded with polydopamine nanoparticles, wherein the extracellular vesicles loaded with the polydopamine nanoparticles are prepared by the preparation method according to claim 2.

8: Extracellular vesicles loaded with polydopamine nanoparticles, wherein the extracellular vesicles loaded with the polydopamine nanoparticles are prepared by the preparation method according to claim 3.

9: Extracellular vesicles loaded with polydopamine nanoparticles, wherein the extracellular vesicles loaded with the polydopamine nanoparticles are prepared by the preparation method according to claim 4.

10: Extracellular vesicles loaded with polydopamine nanoparticles, wherein the extracellular vesicles loaded with the polydopamine nanoparticles are prepared by the preparation method according to claim 5.
